(19) United States
(12) Patent Application Publication
(10) Pub. No.: US 2007/0117864 A1
(43) Pub. Date: May 24, 2007

(54) Composition for preventing and treating hepatoma

(75) Inventors: Kazumi Yagasaki, Tokyo (JP); Yutaka Miura, Tokyo (JP); Daiki Miura, Tokyo (JP); Risto Santti, Turku (FI); Niina Saarinen, Merimasku (FI)

Correspondence Address: OBLON, SPIVAK, MCCLELLAND, MAIER & NEUSTADT, P.C., 1940 DUKE STREET, ALEXANDRIA, VA, 22314, US

(73) Assignee: Tokyo University of Agr. & Tech. TLO Co., Ltd., Koganei-shi (JP)

(21) Appl. No.: 10/572,945

(22) PCT Filed: Dec. 24, 2004
(86) PCT No.: PCT/JP04/19437

§ 371 Date: Dec. 11, 2006

(30) Foreign Application Priority Data
Dec. 26, 2003 (JP) 2003-435930

(51) Int. Cl. A61K 31/365

(52) U.S. Cl. 514/469; 514/22

(57) ABSTRACT

The present invention provides a liver cancer prophylactic and therapeutic composition, and the present invention relates to a liver cancer prophylactic and therapeutic composition characterized in that this composition contains compounds that have a hepatoma proliferation inhibiting effect as active ingredients, these compounds comprising enterolactone (ENL) or plant lignan precursors of the same; a liver cancer prophylactic and therapeutic drug characterized in that this drug comprises compounds that have a hepatoma proliferation inhibiting effect, these compounds being enterolactone (ENL) or plant lignan precursors of the same, and a pharmacologically permissible carrier component; and a functional food product material characterized in that this food product material is formed by mixing compounds that have a hepatoma proliferation inhibiting effect, these compounds being enterolactone (ENL) or plant lignan precursors of the same as functional components with a food product material.

### TECHNICAL FIELD

The present invention relates to a novel liver cancer prophylactic and therapeutic composition, and more particularly to a liver cancer prophylactic and therapeutic drug which contains enterolactone (ENL) or plant lignan precursor of the same as an active ingredient, and a functional food product which contains such components as functional principles. In the technical fields of liver cancer prophylactic and therapeutic drugs and associated functional food products, the present invention is useful for providing a novel liver cancer prophylactic and therapeutic drug, cancer-inhibiting drug, novel functional food product and the like utilizing the high effect on liver cancer proliferation of enterolactone (ENL) which is the principal metabolite in the bodies of mammals of lignans that are widely found in the roots, leaves, stems, seeds and fruits of plants.

### BACKGROUND ART

Lignans are phenol compounds consisting of phenylpropanoid units; such compounds are distributed though in small amounts in all parts of plants such as roots, leaves, stems, seeds and fruits. Like estrogen, lignans undergo enterohepatic circulation, and are released into the urine as gluconid conjugates or sulfate conjugates (Non-patent Reference 1). There have been reports that secoisolariciresinol diglycoside (SDG), a lignan of flax or flaxseed, these contain large quantities of such lignans inhibits the aggravation of rat mammary cancer cells caused by dimethylbenzanthracene (DMBA) (Non-patent References 2, 3, 4 and 5); furthermore, there have also been reports that the ingestion of rye containing lignans is effective in the early prevention of prostate cancer (non-Patent References 6 and 7). However, there have been no research reports in the past concerning any effect of lignans on liver cancer.

[Non-Patent Reference 1] Axelson M, Setchell K D, The excretion of lignans in rats—evidence for an intestinal bacterial source for this new group of compounds. FEBS Lett. 1981 Jan. 26; 123(2):337-42.

[Non-Patent Reference 2] Serraino M, Thompson L U. The effect of flaxseed supplementation on early risk markers for mammary carcinogenesis. Cancer Lett. 1991 November; 60(2):135-42.

[Non-Patent Reference 3] Serraino M, Thompson L U. The effect of flaxseed supplementation on the initiation and promotional stages of mammary tumorigenesis. Nutr Cancer. 1992; 17(2):153-9.

[Non-Patent Reference 4] Thompson L U, Seidl M M, Rickard S E, Orcheson L J, Fong H H. Antitumorigenic effect of a mammalian lignan precursor from flaxseed. Nutr Cancer. 1996; 26(2):159-65.

[Non-Patent Reference 5] Thompson L U, Rickard S E, Orcheson L J, Seidl M M. Flaxseed and its lignan and oil components reduce mammary tumor growth at a late stage of carcinogenesis. Carcinogenesis. 1996 June; 17(6):1373-6.

[Non-Patent Reference 6] Landstrom M, Zhang J X, Hallmans G, Aman P, Bergh A, Damber J E, Mazur W, Wahala K, Adlercreutz H. Inhibitory effects of soy and rye diets on the development of Dunning R3327 prostate adenocarcinoma in rats. Prostate. 1998 Aug. 1; 36(3):151-61.

[Non-Patent Reference 7] Zhang J X, Hallmans G, Landstrom M, Bergh A, Damber J E, Aman P, Adlercreutz H. Soy and rye diets inhibit the development of Dunning R3327 prostatic adenocarcinoma in rats. Cancer Lett. 1997 Mar. 19; 114(1-2):313-4.

### DISCLOSURE OF THE INVENTION

Under such conditions, based on the prior art described above, the present inventors conducted various studies concerning the effects of lignans and various compounds constituting metabolites of lignans on liver cancer. As a result of this research, the inventors discovered that enterolactone (ENL), which is the principal metabolite of hydroxymatairesinol (HMR) in mammals, a lignan present in spruce trees, has a high inhibitory effect on liver cancer proliferation. Conducting further research, the present inventors perfected the present invention. It is an object of the present invention to provide a novel liver cancer prophylactic and therapeutic composition, liver cancer prophylactic and therapeutic drug, and functional food product.

The present invention that is used to solve the above-mentioned problems is constructed from the following technical means:

(1) A component or composition for inducing apoptosis in hepatoma cells, characterized in that the component or composition contains enterolactone (ENL) or its plant lignan precursor compounds as active ingredient(s), and has the characteristics of 1) inducing apoptosis in hepatoma cells, 2) arresting the cell cycle of hepatoma cells, and 3) ameliorating lipid metabolism abnormalities in living body.

(2) The component or composition according to the above-mentioned (1), wherein the above-mentioned compounds are enterodiol (END), secoisolariciresinol (SECO), syringaresinol, arctigenin, lariciresinol, pinoresinol or sesamin.

(3) A drug for inducing apoptosis in hepatoma cells, characterized in that the drug comprises the component or composition defined in the above-mentioned (1) or (2), and a pharmacologically permissible carrier component.

(4) A functional food product material or food product for ameliorating lipid metabolism abnormalities in living body, characterized in that the food product material or food product contains the component or composition defined in the above-mentioned (1) or (2) as a functional component.

(5) The functional food product material or food product according to the above-mentioned (4), characterized in that the above-mentioned functional food product material or food product is a nutritional supplement food product, nutritional agent, therapeutic food product or health food.

Next, the present invention will be described in greater detail.

The present invention relates to a liver cancer prophylactic and therapeutic composition characterized in that this composition contains enterolactone (ENL) or its plant lignan precursor of the same as active ingredients, and more particularly relates to a liver cancer prophylactic and therapeutic drug and functional food product. Enterolactone (ENL) used as an active ingredient in the present invention is a plant-lignan metabolite which is known as a type of mammalian lignans; for example, this compound is known as the principal metabolite of hydroxymatairesinol (HMR) which is a plant lignan originating in spruce trees. Enterolactone is preferably used in the present invention. However, the present invention is not limited to ENL; plant lignan precursors of the same may also be used.

In the present invention, enterodiol (END), secoisolariciresinol (SECO), secoisolarisiresinol diglycoside (SDG), syringaresinol, arctigenin, lariciresinol, pinoresinol and sesamin may be used as the above-mentioned plant lignan precursors. These plant lignan precursors are converted into enterolactone, which is a metabolite of these precursors, by enteric bacteria via a specified metabolic pathway, so that the inhibitory effect of enterolactone on hepatoma proliferation is manifested.

Plant lignan precursors are converted into the metabolite enterolactone by enteric bacteria in the living body. For example, in the process of metabolism, sugars are removed from SDG so that SECO is produced, and this SECO is converted into ENL via END. Matairesinol (MR) is converted directly into ENL.

In the past, methods in which prophylaxis against human cancers, certain types of noncancerous hormone-dependent disorders and/or cardiac disorders or the like has been achieved by a process in which the concentration of enterolactone or other metabolites of hydroxymatairesinol in human blood serum was increased by administering hydroxymatairesinol, a known as a lignan present in spruce trees to humans have been reported (Japanese Patent Republication No. 2002-541158).

What has actually been investigated in such cases, however, is for example the effect on breast cancer, prostate cancer and colon cancer; there have been no investigations regarding liver cancer. Especially, it has not been known in the past whether methods in which the concentration of enterolactone or other metabolites of hydroxymatairesinol in human blood serum is increased by administering hydroxymatairesinol to humans would have a hepatoma inhibitory effect on hepatoma proliferation.

The above-mentioned enterolactone (ENL) and plant lignan precursors of the same used in the present invention are all universally known compounds; for example, in the case of enterolactone (ENL), commercially marketed products may be used, or this enterolactone may be synthesized by a universally known method. Besides methods in which the active ingredient enterolactone (ENL) is directly administered to humans, the present invention also includes methods in which plant lignin precursors of enterolactone are directly administered to humans. In the present invention, drug compositions used in such methods are included in the scope of the invention. For instance, compositions comprising a liquid or solid material in which an effective dose of the above-mentioned active ingredient is concentrated may be cited as examples of the composition of the present invention. Drug compositions of the present invention may be formulated by mixing for example arbitrary components such as pharmacologically permissible carriers and the like in addition to the above-mentioned active ingredients; there are no particular restrictions on the method, drug configuration or the like that is used. In the present invention, furthermore, functional food product materials or functional food products of arbitrary types and configurations such as food additives, nutritional supplement food products, nutritional agents, therapeutic food products, nutrition-reinforcing food products, health foods and the like can be manufactured by adding specified amounts of the above-mentioned active ingredients as functional components.

In the present invention, for instance, 1 to 10 mg/kg of body weight may be cited as an example of an effective amount of enterolactone (ENL) that is added to a drug composition. However, the present invention is not limited to such an amount. Furthermore, the amount of the above-mentioned active ingredients added to the functional food product or the like can be arbitrarily set in accordance with the type of food product and the like. Moreover, the safety of enterolactone (ENL) as an in vivo metabolite originating in nature or plant lignans that are precursors of this enterolactone with respect to the living body has been confirmed in many studies conducted in the past.

In the past, prophylactic methods against human cancers, non-cancerous hormone-dependent disorders and/or cardiac disorders using a procedure to increase the concentration of enterolactone or other metabolites in human blood serum were used. In actuality, however, what has been investigated in regard to cancer is only specified cancers such as breast cancer, prostate cancer and colon cancer; absolutely nothing is known regarding the effectiveness of such methods against other cancers. In the present invention, as a result of the performance of actual tests on the effectiveness of enterolactone and the like uninvestigated in the past on liver cancer, the effectiveness of this substance has been demonstrated, so that the effectiveness of enterolactone and the like against liver cancer has been confirmed for the first time by special experiments performed by the present inventors. In this field, even if certain components are effective against for example breast cancer, prostate cancer and colon cancer, it is not necessarily the case that these components will be similarly effective against liver cancer. Special experiments must be performed in order to elucidate this, and under conditions in which the effectiveness of enterolactone and the like against liver cancer has not been demonstrated, it is difficult to predict an inhibitory effect of enterolactone and the like on hepatoma proliferation. It is clear that the present invention constitutes the selective invention of a prophylactic method for some human cancers known in the past.

The following effects are obtained as a result of the present invention: (1) a novel liver cancer prophylactic and therapeutic composition, liver cancer prophylactic and therapeutic drug composition and a functional food product that have a hepatoma proliferation inhibiting effect can be provided, (2) novel drugs and the like containing enterolactone, which is a metabolite originating from plant lignans, as an active ingredient can be provided, (3) this compound has a high inhibitory effect on hepatoma proliferation at a low dosage, and (4) this compound can also ameliorate abnormalities in hepatic cancerous lipid metabolism.

### BRIEF DESCRIPTION OF THE DRAWINGS

FIG. 1 shows the results of an analysis of the effect of enterolactone on cell cycle;

FIG. 2 shows the results of an analysis of the effect of enterolactone on apoptosis;

FIG. 3 shows the results of a comparison of effect of enterolactone on solid tumor sizes over days;

FIG. 4 shows the serum cholesterol levels; and

FIG. 5 shows the excretion of steroids into feces and bile acids excretions.

### BEST MODE FOR CARRYING OUT THE INVENTION

Next, the present invention will be described in concrete terms on the basis of examples. However, the present invention is not limited in any way by the examples described below.

### Example 1

In the present example, the anti-tumor effect of ENL on AH109A cells was investigated in order to analyze (in vitro/Ex vivo) the effect of the ENL on the proliferation and tumor invasion of hepatoma cells.

1. Materials and Methods

(1) Materials

The chemical structural formula of ENL used in the present example is shown in Formula 1. 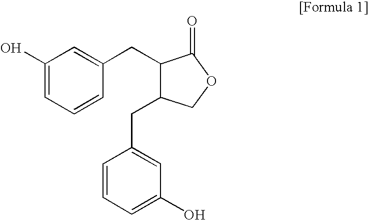
(2) Culture of Hepatoma Cells

Rat ascites hepatoma AH109A cells (obtained from Institute of Development, Aging and Cancer, Tohoku University, Sendai, Japan) were maintained by continuous intra-abdominal transplantation in the peritoneal cavity of male Donryu rats (NRC Haruna, Gumma, Japan). The AH109A cells were purified from the rat ascites when sufficient ascites had accumulated in the abdomen. The ascitic fluid was centrifuged for 10 minutes at 1000 rpm (190×g), 4° C., to remove the supernatant. The same volume of a hemolyzing buffer solution (0.16M N₄Cl:0.17M Tris(hydroxymethyl)aminomethane=9:1, pH 7.2 (all purchased from Wako Pure Chemical Industries, Osaka Japan)) was added to the sedimented cells, pipetting this to form a suspension, and then the red blood cells were destroyed by allowing this preparation to stand for approximately 20 minutes on ice.

Furthermore, above operation step of removing the supernatant and then eliminating the blood cells after centrifugal separation of the preparation for 10 minutes at 1000 rpm (190×g), 4° C. was performed twice, and the resulting preparation was washed twice with a phosphate buffer solution [phosphate-buffered saline (−) (PBS (−)), prepared by dissolving 8 g of NaCl, 0.2 g of KCl, 0.2 g of KH₂PO₄ and 2.9 g of Na₂HPO₄.12H₂O (all purchased from Wako Pure Chemical Industries, Osaka, Japan) in 1 L of MQ water, and adjusting the pH to 7.4]. The purified AH109A cells were inoculated into a culture dish (6 cm in diameter, NUNC, Roskilde, Denmark) using an Eagle MEM medium (Nissui Pharmaceutical Co., Ltd., Tokyo, Japan) containing 10% heat-inactivated calf serum (CS, JRH Bioscience, Lenexa, Kans., USA)(10% CS/MEM) at a concentration of 1×10⁶ cells per dish, and were cultured. The CS was inactivated in a water bath for 30 minutes at 56° C. prior to use.

(3) Measurement of Proliferative Activity of Hepatoma Cells

The proliferative activity of the AH109A cells was evaluated by measuring the incorporation of [³H]thymidine (20 Ci/mmol, New England Nuclear, Boston, USA) into acid-insoluble fraction, namely, DNA [Yagasaki K., Tanabe T., Ishihara K., and Funabiki R., (1992) Modulation of the proliferation of cultured hepatoma cells by urea cycle-related amino acids. In: Murakami, H., Shirahata S., and Tachibanana H., (ed.) Animal Cell Technology: Basic and Applied Aspects. Vol. 4 (pp. 257-263). Kluwer Academic Publisher, Dordrecht/Boston/London].

Specifically, the AH109A cells were inoculated in a 48-well plate (Nunc) at a cell density of 2.5×10⁴ cells in 400 μl of 10% CS/MEM per well, and were cultured for 20 hours. [³H]thymidine was added to each well at the rate of 0.15 μCi/well, and cultured for another 4 hours in a CO₂ incubator. At the end of cell culture, 1M ascorbic acid (50 μl/well, Wako Pure Chemical Industries) was added at the rate of 50 μl/well, and the media in the respective wells were recovered in a Maruemu tube (Maruemu Corporation Co., Ltd.). The 48-well plate was washed twice with PBS (−), and the wash solution was also recovered in Maruemu tubes. The resulting preparations were centrifugally separated for 5 minutes at 1500 rpm (400×g), 4° C., and the supernatant was removed by suction.

The plate was washed twice with 400 μl of 10% TCA; the wash solution was then recovered in Maruemu tubes, and was again subjected to centrifugally separation for 5 minutes at 3000 rpm (1500×g), 4° C., and then solubilized in 0.2N NaOH in 0.1% SDS. was added to the 48-well plate and the cells remaining in the Maruemu tubes at the respective rates of 150 μl and 50 μl, and these were allowed to stand for 30 minutes inside an incubator at 37° C., to dissolve the cells completely. The dissolved cells solution was transferred to a mini-vial together with both the 48-well plate and the Maruemu tubes being combined. 5 ml of an NT scintillator (700 ml of toluene, 300 ml of nonion, 4 g of DPO) was added, and 150 μl of 1N HCl was further added thereto, and it was confirmed that the liquid of the preparation thoroughly shaken had become transparent; then, the radioactivity of the preparation was measured with a liquid scintillation spectrophotometer (LS6500; Beckman, Fullerton, Calif., USA). The percentage (%) relative to a control value of 100 was calculated, and this value was taken as an index of the proliferative activity.

(4) Measurement of Invasive Activity of Hepatoma Cells

The tumor invasion activity was measured using a partially modified version (Miura Y., Shiomi H., Sakai F., and Yagasaki K. 1997 Assay systems for screening food components that have anti-proliferative and anti-invasive activity to rat ascites hepatoma cells: In vitro and ex vivo effects of green tea extract. Cytotechnology 23: 127-132) of the in vitro invasion assay method of Akedo et al (Akedo H, Shinkai K, Mukai M, Mori Y, Tateishi R, Tanaka K, Yamamoto R, Morishita T. Interaction of rat ascites hepatoma cells with cultured mesothelial cell layers: a model for tumor invasion. Cancer Res. 1986 May; 46(5):2416-22).

Mesothelial cells (M-cells) directly prepared from rat mesenteries to culture were cultured for 7 to 10 days until a confluent monolayer was formed, and subsequently, the medium was changed to 3 ml of the respective experimental media. AH109A cells were overlayed onto the mesothelial cells at the density of 2.4×10⁴ cells, and were gently agitated, and were cultured in a CO₂ incubator. After 24 hours, the cells were washed with PBS (−), and the cells were fixed with 0.25% glutalaldehyde/PBS (−). The numbers of cells and numbers of colonies of the AH109A cells that had infiltrated beneath the mesothelial cell monolayer were counted in 10 places of 2-mm square divisions selected at random for 1 dish, and the mean values of the numbers obtained by calculating these counts per square centimeter were taken as an index of the tumor invasion activity.

(5) Preparation of Mesothelial Cells (M-Cells) from Mesenteric Membrane

Male Donryu rats (4 to 10 weeks of age) were anesthetized by intraperitoneal injection of Nembutal at the dose of 5 mg/0.1 ml/100 g of body weight, and blood was collected by cutting the carotid artery. Subsequently, following disinfection using a hibiten solution, the abdomens of the animals were opened in a clean bench, and the mesenteric membranes were removed one by one and washed with PBS (−), and were then recovered in another 15 ml of PBS (−). The same amount of 0.5% trypsin in PBS (−) was added, and the preparation was agitated for 20 minutes at 37° C. Subsequently, 5 ml of a 10% CS/MEM was added, to stop the trypsin reaction. Then, the preparation was subjected to pipetting by means of a Komagome Pipette, and filtrated using a metal mesh. The filtrate was centrifuged for 10 minutes at 1500 rpm (440×g), 4° C., and was washed with PBS (−). The collected cells were inoculated into a 6 cm dish with an attached grid (2 mm square) at the density of 1.5 to 2.0×10⁵ cells. The medium of 10% CS/MEM was used, and the medium was changed on the day following collection and every other day thereafter.

(6) Measurement of Proliferative Activity of M-Cells

The M-cells were cultured until they reached a sub-confluent state; then, the cells were washed with PBS (−), and the cells were dispersed by adding 1 ml of 0.1% trypsin, the cells were recovered after the trypsin reaction was stopped with 10% CS/MEM, and the cells were then inoculated in a 48-well plate at the density of 1.25×10⁵ cells/400 μl/well, and were cultured for 24 hours. After the cells were thoroughly adhered, the medium was removed, and the sample was added. Then, the cells were cultured for 20 hours at 37° C. After each experimental medium was added thereto so that the ultimate amount added was 400 μl/well, the cells were cultured for 20 hours. Subsequently, [³H]thymidine (NEN, Boston, Mass., USA) was added to each well at the rate of 0.15 μCi/well, and the cells were cultured for an additional 4 hours in a CO₂ incubator. Subsequently, the proliferation activity was measured as above-mentioned in (3).

(7) Cell Cycle Analysis of Hepatoma Cells

The effects of 6.25 μM and 12.5 μM ENL on the cell cycle of AH109A cells were analyzed. The AH109A cells were inoculated into a 6-well plate (NUNC) at the density of 2.5×10⁵ cells per well, and the cells were cultured for 0, 24 and 48 hours at 37° C. in a CO₂ incubator. The AH109A cells cultured under the respective treatment conditions were recovered in 2-ml sampling tubes (Nippon Genetex, Tokyo); these cells were centrifuged for 5 minutes at 1000 rpm (190×g), 4° C., and then washed twice with PBS (−). Subsequently, 300 μl of a PI solution (1 mg propidium iodide (SIGMA)/20 ml 0.1% Triton X-100, 0.1% sodium citrate (Wako Pure Chemical Industries)) was added, and the preparation was dyed by allowing the preparation to kept quietly on ice for 30 minutes with the light blocked. The cell cycle was analyzed with a flow cytometer (EPICS ELITE ESP; Beckman-Coulter, Hialeah, Fla., USA).

(8) Analysis of Apoptosis of AH109A Cells by Flow Cytometry Using the Annexin V/PI Double Staining Method

The analysis was performed using an ANNEXIN V FITC kit (IMMUNOTECH, Marseille, France). AH109A cells were inoculated at the density of 1×10⁶ cells with 3 ml of the experimental medium in a dish with 6 cm in diameter (NUNC), and the cells were cultured for 0, 3 and 6 hours in a CO₂ incubator at 37° C. The AH109A cells cultured under the respective treatment conditions were recovered, centrifuged for 5 minutes at 4° C. at 1000 rpm (90×g), and washed once with PBS (−). Subsequently, the cells was suspended in 490 μl of a binding buffer, and 5 μl of a PI solution and 5 μl of ANNEXIN V FITC, and these substances were gently mixed. The cells were stained by allowing the preparation to stand quietly for 10 minutes on ice with the light blocked. The apoptosis of the cells was analyzed using a flow cytometer (EPICS ELITE ESP; Beckman-Coulter, Hialeah, Fla., USA).

(9) Statistical Analyses

The statistical analyses were performed by one-way analysis of variance, followed by Tukey-Kramer multiple comparisons test.

2. Experimental Results

(1) The Effect of ENL on AH109A Cells and Normal Cells

ENL showed a dose-dependent inhibitory effect on tumor invasion of AH109A cells. At ENL concentrations of 25 μM or greater, it was impossible to analyze the tumor invasion activity, since cell toxicity was seen in the M-cells seen in the assay system for tumor invasive activity. Accordingly, analyses thereafter thereof was were performed at concentrations below 12.5 μM. ENL also strongly suppressed the proliferation of a cancer cell line of AH109A cells its effect on normal cells was also investigated using M-cells as normal cells, and the results were compared. As a result, ENL was found to inhibit the proliferation of M-cells in a dose-dependent manner in vitro; however, ENL showed a stronger inhibitory effect against AH109A cells than normal M-cells, and it was clear that the proliferation of AH109A cells was almost completely suppressed by ENL at a concentration of 50 μM.

In order to clarify whether mechanisms of cell prolification inhibitory is due to suppression of cell division or induction of apoptosis and/or necrosis, by which ENL suppresses the proliferation of AH109A cells, mechanisms of cell prolification on AH109A cells by which ENL suppresses was investigated by analyzing the cell cycle by PI staining and observing the inductivity of apoptosis by means of ANNEXIN V FITC/PI.

(2) Analysis of Cell Cycle

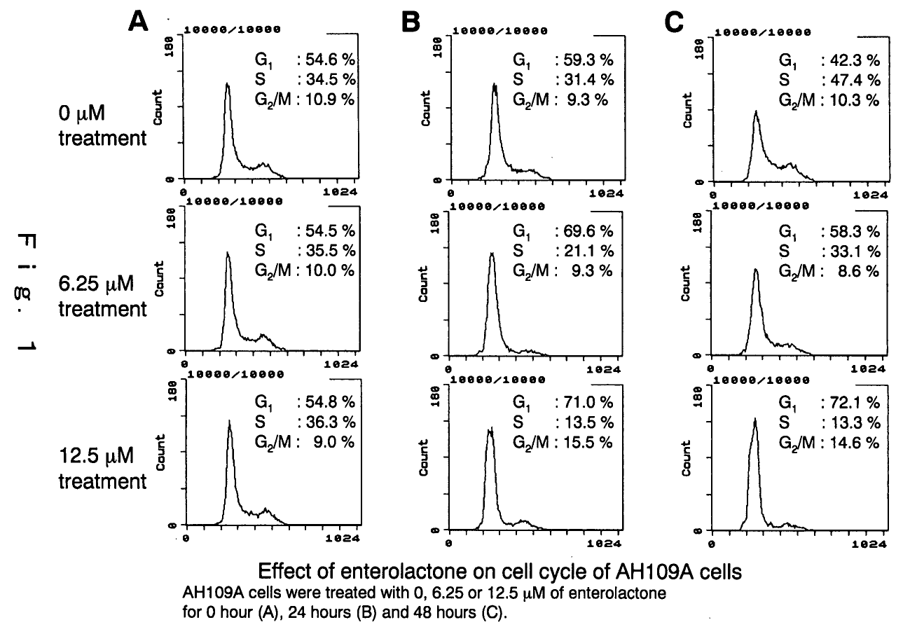

FIG. 1 shows results of the analysis. On graph A indicating ENL treatment for 0 hours, an ENL treatment at various concentrations seemed to have no effect on the cell cycle. However, on graph B indicating ENL treatment for 24 hours, the ENL-treated cells showed an increase in the proportion of the G1 phase and a decrease in the proportion of the S phase were observed as compared to the control of the (0 μM) treatment group.

(3) Regarding Apoptosis

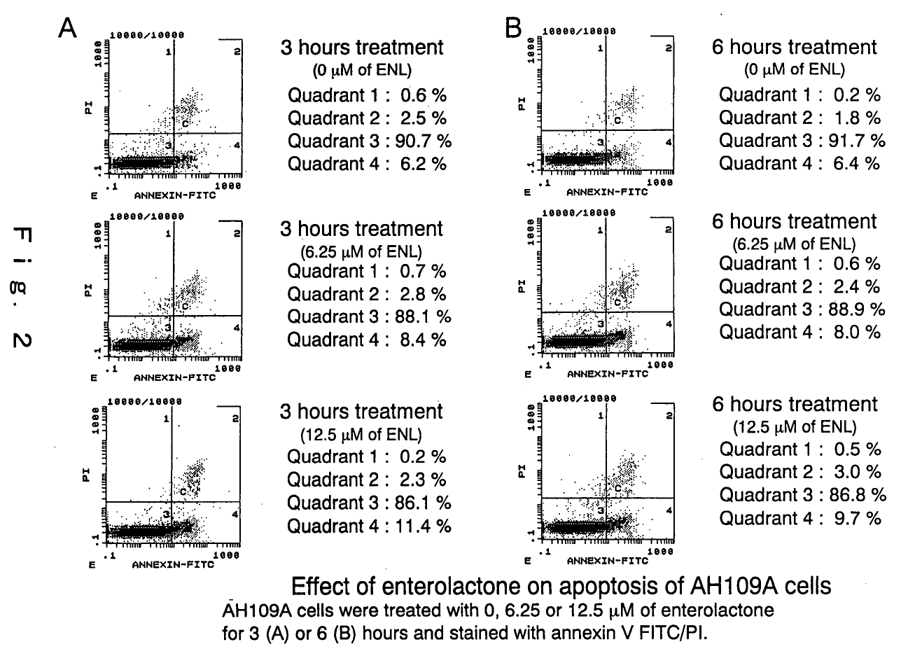

The results of the analysis are shown in FIG. 2. An investigation was conducted over time as to whether or not ENL at concentrations of 6.25 μM and 12.5 μM caused the induction of apoptosis to occur in AH109A cells. In AH109A cells treated for 3 hours without ENL (0 μM), the proportion of the third quadrant was 90.7%, that of the fourth quadrant was 6.2%, and that of the second quadrant was 2.5%. However, in AH109A cells treated for 3 hours with 6.25 μM ENL, the proportion of the third quadrant was 88.1%, that of the fourth quadrant was 8.4%, and that of the second quadrant was 2.8%; furthermore, in AH109A cells treated for 3 hours with 12.5 μM ENL, the proportion of the third quadrant was 86.1%, that of the fourth quadrant was 11.4%, and that of the second quadrant was 2.3%. As a result of treatment with ENL, the proportion of live cells decreased in a dose-dependent manner, and the proportion of cells in the early stage of apoptosis increased. Similar results were also shown for AH109A cells treated for 6 hours. Moreover, the proportion of cells showing secondary necrosis also increased to some extent.

### Example 2

In the present example, in order to analyze effect of ENL on solid tumor growth, metastasis and cancerous hyperlipemia in vivo, the animals were actually fed ENL-supplemented diets, and the effects on tumor growth, metastasis and cancerous cachexia in a cancer-bearing rat model were investigated.

1. Materials and Methods

(1) Diets of Animals

Male Donryu rats of four weeks old were purchased from NRC Haruna, Gumma, Japan, and were preliminarily fed for 6 days in quintuple cages in a room with a controlled environment with light period of 8:00 to 20:00, room temperature of 22±1° C., and relative humidity of 60±5%. For the first 3 days, the animals were fed ad libitum with a stock pellet diet (CE-2; CLEA Japan, Tokyo), and for the next 3 days, they were fed a basal diet containing 20% milk casein (20C) (Table 1) ad libitum together with tap water in a glass container. Following the completion of this preparatory raising, the rats were divided into three groups (11 animals per group) with equal average body weights of the respective groups were equal, and 1.0×10⁷ of AH109A cells suspended in PBS (−) were transplanted in the back of all the rats. From the time immediately after transplantation, in the respective experimental groups, the animals were fed the experimental diets shown in Table 2.

During the experimental period, the body weight, food intake and solid tumor size (sum of length, width and height) were measured everyday. On the day that the animals were killed, animals were deprived of experimental diets at 9:00, but freely given water until they were killed 4 hours later. Blood was collected from carotid, and the liver and solid tumors were excised. The collected blood was allowed to stand for approximately 2 hours at room temperature, and then centrifuged for 10 minutes at 3000 rpm (1750×g), 4° C., to obtain serum. This blood serum was frozen and stored at −20° C. The livers and solid tumors were washed with physiological saline, blotted on filter paper, and weighed. Furthermore, feces from the 19^{th} to 21^{st} days after transplantation were collected, and these feces were frozen and stored at −20° C. for the measurement of the amounts of steroids excreted from the body.

TABLE 1
Composition of experimental diets

| Ingredient (g/kg) | Control (20 C.) |
|---|---|
| Cornstarch^{a} | 397.5 |
| Casein^{b} | 200.0 |
| α-Cornstarch^{a} | 132.0 |
| Sucrose^{c} | 100.0 |
| Soybean oil^{d} | 70.0 |
| Cellulose powder^{b} | 50.0 |
| Mineral mixture (AIN-93G)^{a,e} | 35.0 |
| Vitamin mixture (AIN-93)^{a,f} | 10.0 |
| L-Cystine^{g} | 3.0 |
| Choline bitartrate^{h} | 2.5 |

^{a}Nihon Nosan Kogyo Co., Ltd., Yokohama, Japan
^{b}Oriental Yeast Co., Tokyo, Japan
^{c}Mitsui Sugar Co., Ltd., Tokyo, Japan
^{d}Miyazawa Yakuhin Co., Ltd., Tokyo, Japan
^{e}AIN-93G composition
^{f}AIN-93 composition
^{g}Ajinomoto Co., Inc., Tokyo, Japan
^{h}Wako Pure Chemical Industries, Ltd., Osaka, Japan

TABLE 2
Composition of experimental diets

| | | | Entero- |
|---|---|---|---|
| | Control | Enterolactone | lactone |
| Ingredient (g/kg) | (20 C.) | (10 ppm) | (100 ppm) |
| Enterolactone | — | 0.01 | 0.10 |
| Cornstarch^{a} | 397.50 | 397.49 | 397.40 |
| Casein^{b} | 200.00 | 200.00 | 200.00 |
| α-Cornstarch^{a} | 132.00 | 132.00 | 132.00 |
| Sucrose^{c} | 100.00 | 100.00 | 100.00 |
| Soybean Oil^{d} | 70.00 | 70.00 | 70.00 |
| Cellulose Powder^{b} | 50.00 | 50.00 | 50.00 |
| Mineral Mixture (AIN-93G)^{a,e} | 35.00 | 35.00 | 35.00 |
| Vitamin Mixture (AIN-93)^{a,f} | 10.00 | 10.00 | 10.00 |
| L-Cystine^{g} | 3.00 | 3.00 | 3.00 |
| Choline Bitartrate^{h} | 2.50 | 2.50 | 2.50 |

^{a}Nihon Nosan Kogyo Co., Ltd., Yokohama, Japan
^{b}Oriental Yeast Co., Tokyo, Japan
^{c}Mitsui Sugar Co., Ltd., Tokyo, Japan
^{d}Miyazawa Yakuhin Co., Ltd., Tokyo, Japan
^{e}AIN-93G composition
^{f}AIN-93 composition
^{g}Ajinomoto Co., Inc., Tokyo, Japan
^{h}Wako Pure Chemical Industries, Ltd., Osaka, Japan

(2) Measurement of Changes in Solid Tumors

The length, width and height of the solid tumors were measured, and the sums of these values were used as an index of solid tumor size.

(3) Measurement of Serum Lipid Levels

Serum cholesterol (Ch) and triglyceride (TG) concentration was measured. Total cholesterol (T-Ch) level, the high-density-lipoprotein (HDL-Ch) level (which is the supernatant fraction obtained by precipitating very-low-density-lipoprotein+low-density-lipoprotein ((VLDL+LDL)-Ch fractions) by the precipitation method using phosphotungstic acid were measured. The (VLDL+LDL)-Ch level was taken as the difference between the T-Ch and the HDL-Ch levels. Atherogenic index (AI: (VLDL+LDL)-Ch/HDL-Ch ratio) was calculated as an index of atherogenesis.

(4) Measurement of Serum Lipid Peroxide Levels

LPO levels were determined by measuring thiobarbituric acid-reactive substances (TBARS) by the Yagi fluorescence method using a lipid peroxide test Wako (Wako Pure Chemical Industries).

(5) Measurement of Lipid Levels in the Liver

1) Extraction of Total Lipids

Total lipids were extracted from the liver for the measurement of lipid levels by the method of Folch et al. (FOLCH J, LEES M, SLOAN-STANLEY G H, A simple method for the isolation and purification of total lipids from animal tissues, J. Biol. Chem. 226: 497-509, 1957). Approximately 0.5 g of liver tissue was weighed out, this tissue was homogenized using a Polytron homogenizer (Type PT10/35, Kinematica, Switzerland) in 5 ml of methanol; then, 10 ml of chloroform was added and mixed, the resulting preparation was allowed to stand overnight, and then filtered. The residue was further washed with a mixture of chloroform and methanol (2:1), and both solutions were combined to produce a 25 ml sample, which was taken as the lipid extract.

2) Measurement of Total Cholesterol Level

The Ch content was measured by the Zak method (Zak B., Simple rapid microtechnic for serum total cholesterol, Am. J. Clin. Path. 27, 583-588, 1957). The lipid extract (2 ml) was placed in a screw-capped test tube, dried, and then saponified for 1 hour at 45° C. with 3 ml of a 50% (w/v) ethanol solution of potassium hydroxide. This preparation was diluted by adding 3 ml of distilled water; then, Ch was extracted by adding 3 ml of n-hexane and shaking. An aliquot (2 ml) of the hexane layer was placed into a test tube and dried; then, 2 ml of a 0.08% acetic acid solution of ferric chloride (prepared by dissolving 1.33 g of ferric chloride in acetic acid to form 1 L of solution) and 2 ml of sulfuric acid were added. The resulting mixture was agitated and this preparation was allowed to stand until cool; then, the absorbance at 560 nm thereof was measured with Ch as a standard.

3) Measurement of Triglyceride Level

The TG level was measured by the Van Handel method (VAN HANDEL E., Suggested modifications of the micro determination of triglycerides, Clin. Chem., 7: 249-51, 1961). The lipid extract was placed in a screw-capped test tube, and dried. Next, 0.5 g of zeolite (Wako Pure Chemical Industries) and 10 ml of chloroform were added, and the resulting mixture was shaken to remove phospholipids (PL). Subsequently, this preparation was filtered, and 2 ml of the filtrate was placed in a screw-capped test tube, dried, and then saponified for 20 minutes at 65° C. by adding 0.5 ml of a 0.4% (w/v) ethanol solution of potassium hydroxide. The reaction was stopped by adding 0.5 ml of 0.2N sulfuric acid. After the ethanol in the test tube was removed in a boiling water bath, 0.005 ml of 0.5% sodium meta-periodate was added to induce oxidative decomposition. After 10 minutes, the reaction was stopped by adding 0.05 ml of 5% sodium hydrogensulfite. 5 ml of a mixture of chromotropic acid-sulfuric acid solution [prepared by dissolving 2.24 g of chromotropic acid dehydrate (DOJINDO, Kumamoto) in distilled water to form 200 ml of solution, and then adding 900 ml of 24 N sulfuric acid under ice cooling] was added to this, and the resulting preparation was heated for 30 minutes in a boiling water bath. After cooling, the absorbance at 570 nm thereof was measured using tripalmitin as a standard.

(6) Measurement of Fecal Steroids

The amounts of fecal steroids, neutral sterol (NS) and bile acid (BA), excreted into feces were measured. The feces were dried at 60° C., and the dry weight was measured; then, the feces were pulverized using a mixer and a mortar and pestle. Approximately 100 mg of feces was precisely weighed and placed in a screw-capped test tube, and then was saponified for 1 hour at 70° C. by adding 1.5 ml of 4 N potassium hydroxide solution and 1 ml of ethanol. The NS was extracted by transferring the n-hexane layer to a test tube by adding 3 ml of n-hexane to a test tube, shaking this mixture and then subjecting to centrifugal separation at 3000 rpm (1750×g) for 10 minutes. The same operation was performed three times. Subsequently, with 2.5 mol of distilled water to dilute potassium hydroxide concentration thereof to 1.2 N, and the mixture was subjected to a high-pressure treatment (autoclaved) for 3 hours at 121° C., to deconjugate the conjugated BA. After the preparation was allowed to stand until cool, BA was extracted by adding 3 ml of diethyl ether after adjusting the pH of the solution to 1 with 0.8 ml of hydrochloric acid, and the resulting mixture was shaked; then, subjecting this preparation to centrifugal separation for 10 minutes at 3000 rpm (1750×g), to transfer the diethyl ether layer to a test tube. This operation was performed three times.

The n-hexane extract was dried; then, was added with 0.4 ml of isopropanol and 1.6 ml of 10% Triton X-100. to dissolve the NS. An aliquot (0.24 ml) of this solution was sampled, and was subjected to measurement of NS using a cholesterol C test Wako. The diethyl ether extract was dried; then was added with dissolved in 2 ml of methanol to dissolve the BA. An aliquot (0.02 ml) of this solution was sampled, and was subjected to measurement of BA using a total bile acid test Wako by means of an enzyme method using 3α-hydroxysteroid dehydrogenase. This was indicated by the total amount excreted over the final two days.

(7) Statistical Analyses

The statistical analyses were performed a by one-way analysis of variance, followed by Tukey-Kramer multiple comparisons test.

2. Experimental Results

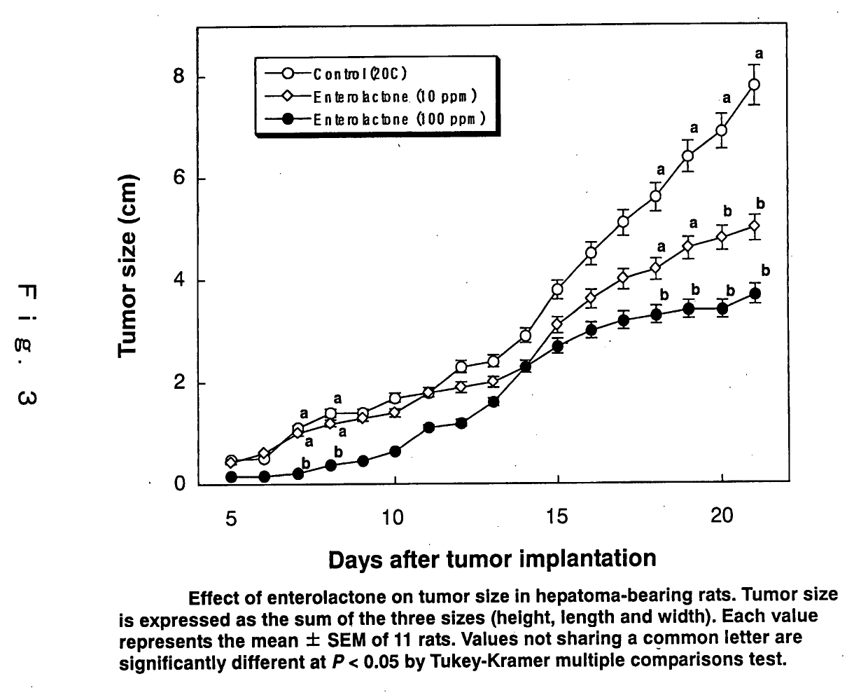

The results of a comparison of solid tumor size are shown in FIG. 3. The formation of solid tumors began to be palpable on the fifth day following hepatoma transplantation. Compared to the control diet group, the ENL ingesting groups, especially the 100 ppm ENL ingesting group, showed a significant suppression of solid tumor sizes on the 7th and 8th days and on the 18th to 21 days following hepatoma transplantation. Next, as shown in Table 3, when tumors metastasized over a period of 21 days following transplantation in the back were compared, the proportion of rats showing metastasized tumors was 27.3% (3 of 11 animals) in the control group, while no metastasis was observed in the ENL diet groups.

Furthermore, the respective groups showed no significant differences in amount of food ingested, liver weight, lipid peroxides or liver lipid levels in the liver; however, body weight showed a significant increase in the 100 ppm ENL ingestion group, and solid tumor weight showed a significant decrease in both ENL ingestion groups (Table 4).

TABLE 3
Effect of enterolactone on metastasis

| | | | Enterolactone | Enterolactone |
|---|---|---|---|---|
| | Control (20 C.) | | (10 ppm) | (100 ppm) |
| Metastasized | 3/11 | (27.3%) | 0/11 (0%) | 0/11 (0%) |
| rats/group | | | | |
| Total number of | 4 | (1, 2, 1) | 0 | 0 |
| metastasis/group | | | | |

TABLE 4
Food intake, body weight gain, liver and hepatoma weights, serum and liver
lipid levels and fecal steroid excretion in hepatoma-bearing rats

| | Control | Enterolactone | Enterolactone |
|---|---|---|---|
| Measurement | (20 C.) | (10 ppm) | (100 ppm) |
| Food intake (g/21 days) | 424 ± 19 | 433 ± 14 | 472 ± 9 |
| Body weight gain (g/21 days) | 146 ± 8^{a} | 158 ± 8^{a} | 171 ± 4^{b} |
| Liver weight (g/rat) | 11.6 ± 0.8 | 10.1 ± 0.5 | 11.3 ± 4.0 |
| Hepatoma weight (g/rat) | 9.6 ± 2^{a} | 3.4 ± 1^{b} | 1.1 ± 0.2^{b} |
| Serum lipid level | | | |
| Total Cholesterol (mmol/L) | 2.52 ± 0.09 | 2.13 ± 0.14 | 2.30 ± 0.12 |
| HDL-Cholesterol (mmol/L) (A) | 1.39 ± 0.09 | 1.45 ± 0.07 | 1.63 ± 0.06 |
| (VLDL + LDL)-Cholesterol (mmol/L) (B) | 1.13 ± 0.16^{a} | 0.68 ± 0.12^{b} | 0.67 ± 0.08^{b} |
| AI (B/A) | 1.00 ± 0.29 | 0.48 ± 0.09 | 0.41 ± 0.05 |
| Triglyceride (mmol/L) | 1.28 ± 0.24 | 0.98 ± 0.06 | 0.93 ± 0.06 |
| TBARS (nmol/ml) | 8.53 ± 0.73 | 7.31 ± 0.37 | 6.87 ± 0.51 |
| Liver lipid level (μmol/g liver) | | | |
| Triglyceride | 38.2 ± 3.61 | 43.8 ± 3.83 | 42.8 ± 7.05 |
| Total cholesterol | 5.13 ± 0.41 | 5.15 ± 0.43 | 5.47 ± 0.40 |
| Steroid excretion | | | |
| Fecal dry weight (g/2 days) | 2.57 ± 0.13 | 2.87 ± 0.18 | 2.99 ± 0.14 |
| Neutral sterols (μmol/2 days) | 20.2 ± 2.3^{a} | 29.5 ± 2.7^{b} | 30.9 ± 2.18^{b} |
| Bile acids (μmol/2 days) | 14.8 ± 1.07^{a} | 18.7 ± 1.49^{ab} | 20.8 ± 1.89^{b} |

Each value represents the mean ± SEM of 11 rats. Values not sharing a common letter are significantly different at P < 0.05 by Tukey-Kramer multiple comparisons test.

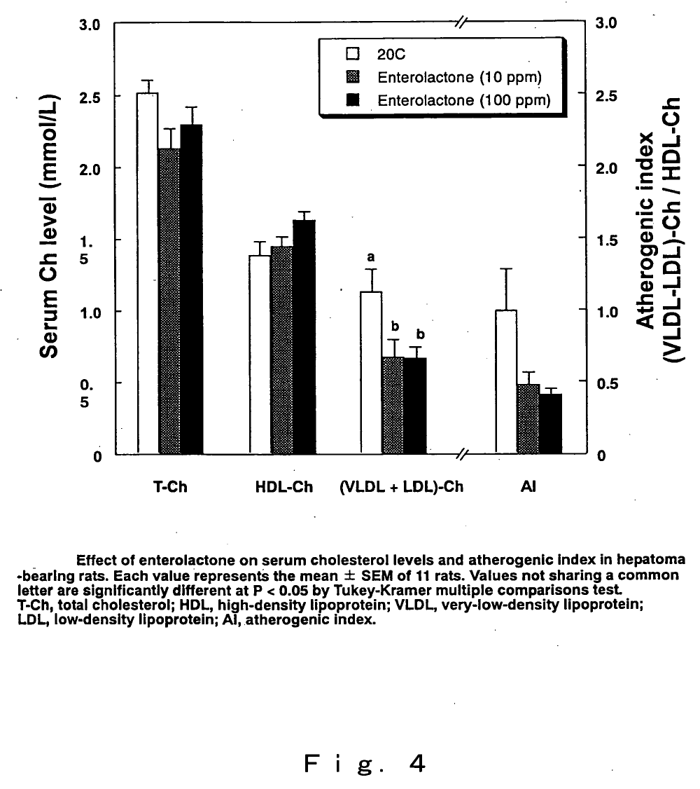

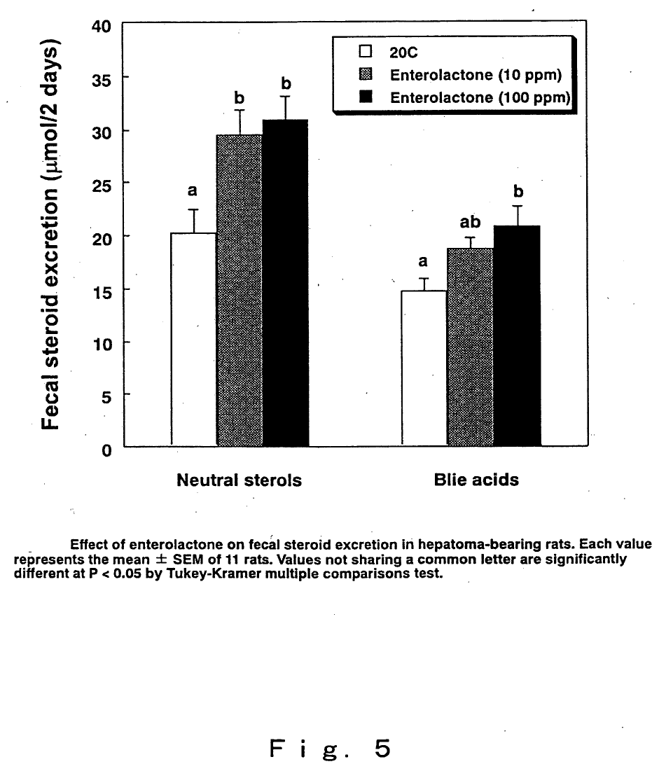

Next, FIG. 4 shows a graph of the cholesterol concentration in the blood serum. The ENL diet groups showed a tendency increase HDL-Ch in the blood serum compared to the control diet group, although the difference was not significant. Conversely, (VLDL+LDL)-Ch decreased significantly in a dose-dependent manner in the ENL diet groups; as a result, AI value, which is the ratio of (VLDL+LDL)-Ch to HDL-Ch, also showed a tendency to decrease. FIG. 5 shows the excretion of neutral sterol and bile acid into the feces. Compared to the control diet group, the ENL diet groups showed a significant and dose-dependent increase in the amounts of neutral sterol and bile acid excreted.

### Example 3

Enterolactone (ENL) as an active ingredient, and a pharmacologically permissible carrier component, were mixed in specified amounts; next, a liver cancer prophylactic and therapeutic drug composition was manufactured by formulating these ingredients.

### Example 4

A functional food product with a hepatoma proliferation inhibiting effect was manufactured by using enterolactone (ENL) as an active ingredient, and mixing other nutritional components with this active ingredient.

### INDUSTRIAL APPLICABILITY

As was described above in detail, the present invention relates to a liver cancer prophylactic and therapeutic composition. The present invention makes it possible to provide a novel liver cancer prophylactic and therapeutic drug composition, a functional food product and the like utilizing the hepatoma proliferation inhibiting effect of enterolactone. In addition to a liver cancer prophylactic and therapeutic drug, the present invention makes it possible to provide for example functional food product materials and functional food products such as food additives, nutritional supplement food products, nutritional agents, therapeutic food products, nutritional supplement food products, health foods and the like that manifest a hepatoma proliferation inhibiting effect.

1. A component or composition for inducing apoptosis in hepatoma cells, characterized in that the component or composition contains enterolactone (ENL) or plant lignan precursor compounds of thereof as active ingredient(s), and has the characteristics of 1) inducing apoptosis in hepatoma cells, 2) arresting the cell cycle of the cells, and 3) ameliorating lipid metabolism abnormalities in living body.

2. The component or composition according to claim 1, wherein said compound is enterodiol (END), secoisolariciresinol (SECO), syringaresinol, arctigenin, lariciresinol, pinoresinol or sesamin.

3. A drug for inducing apoptosis in hepatoma cells, characterized in that the drug comprises the component or composition defined in claim 1 or claim 2, and a pharmacologically permissible carrier component.

4. A functional food product material or food product for ameliorating lipid metabolism abnormalities in living body, characterized in that the food product material or food product contains the component or composition defined in claim 1 or claim 2 as a functional component.

5. The functional food product material or food product according to claim 4, characterized in that the functional food product material or food product is a nutritional supplement food product, nutritional agent, therapeutic food product or health food.
